(19) United States
(12) Patent Application Publication
(10) Pub. No.: US 2006/0136066 A1
(43) Pub. Date: Jun. 22, 2006

(54) Postero-stabilised prosthesis with non-shifting femoral stud

(76) Inventors: Sylvie Plumet, Chaumont (FR); Alexis Vouaux, Poulangy (FR)

Correspondence Address: Edward G. Greive;Renner, Kenner, Greive, Bobak, Taylor & Weber, Fourth Floor, First National Tower, Akron, OH, 44308-1456, US

(21) Appl. No.: 10/829,687

(22) Filed: Apr. 22, 2004

(30) Foreign Application Priority Data
Apr. 24, 2003 (FR) 0305063

(51) Int. Cl. A61F 2/38

(52) U.S. Cl. 623/20.27

(57) ABSTRACT

Full knee prosthesis having a tibial part, a tibial insert and a femoral part comprising two condyles between which extends a femoral stud of a cylindrical shape in order to define an opening into which passes a tibial stud projecting from the tibial insert, the tibial insert having concave upper surfaces in contact with the convex outer surfaces of the condyles, the contact being defined by a contact zone having a central point, characterised in that the curve delimiting the transverse cross-section of the femoral stud is defined so that the central point of the contact zone undergoes a rearward translation (posterior recoil) which decreases according to the angle of flexion from a flexion of about 30° to become zero for an angle of flexion of 80 to 90°, and remains zero to a maximum flexion of 120-130°.

The present invention relates to a knee prosthesis, a so-called postero-stabilised prosthesis. This prosthesis has a femoral part intended to be fixed to a femur, in particular by anchoring means, and a tibial part intended to be fixed to the proximal part of a tibia, in particular also by anchoring means. Between the tibial part and the femoral part an insert (also referred to as a meniscus) is inserted which can be fixed to the tibial part or can be moveable and is usually made from a material which is softer than that of the femoral and tibial parts, such as polyethylene. In its upper part the insert has generally concave surfaces with which two condyles projecting from the femoral part come into sliding or rolling contact. A stud, a so-called tibial stud, projects from the base of the insert, in particular in the perpendicular direction. Between the two condyles of the femoral part an opening, the so-called inter-condyle space, is formed, into which the tibial stud passes. In order to delimit this opening on the posterior side a so-called femoral stud is provided extending transversely to the tibial stud, from one condyle to the other.

During rotation or flexion of the knee the femoral stud comes into contact with the tibial stud, generally from a flexion angle of about 30°.

In current prostheses there is, from this contact value of 30° of flexion, a rearward or posterior recoil of the femoral part which makes it possible to achieve good flexion up to values of 120° to 130°, in particular for obese people. Without this posterior recoil large degrees of flexion such as this would not be possible, especially for obese people.

However, beyond 80 to 90° of flexion the femoral part presents a high risk of rearward recoil possibly leading to considerable strain and ultimately dislocation of the prosthesis.

Furthermore, at the site of contact with the condyles the insert tends to be subject to severe wear.

The present invention aims to overcome these disadvantages of the prior art by proposing a knee prosthesis, a so-called postero-stabilised prosthesis, of which the femoral part has less tendency to shift, the knee prosthesis, in particular its insert, having a greater service life and accepting large degrees of flexion of the order of 120° to 130° or as much as 140°, even for obese people.

In accordance with the invention the so-called postero-stabilised full knee prosthesis, which is formed by a femoral part comprising two condyles between which extends a femoral stud of a cylindrical shape in order to define an opening, by a tibial part having a tibial plate, and by a tibial insert disposed between the femoral part and the tibial plate, a tibial stud, projecting from a piece of the tibial insert, passing into the opening and coming into contact with the femoral stud from a given angle, in particular about 20 to 30°, the tibial insert having concave upper surfaces in contact with the convex outer surfaces of the condyles, the contact defining a contact zone having a central point, is characterised in that the curve delimiting the transverse cross-section of the femoral stud is defined so that the central point of the contact zone undergoes a translation or recoil in the posterior direction for any angle of flexion between about 20° to 30° up to 80 to 90°, and no longer undergoes any posterior recoil for any angle between about 80 to 90° and a maximum flexion of 120-130°.

By providing such kinematics for the condyle-insert point of contact, i.e. an absence of posterior recoil from this point for flexion angles from 80-90° as far as complete flexion (120-135°) but having a posterior recoil up to this value of 80 to 90°, the risk of recoil and shifting of the femoral part and the wearing of the insert are greatly reduced while permitting large degrees of flexion of the knee prosthesis to be achieved, in particular for obese people. Thus a more long-lasting prosthesis is obtained which is safer and which more precisely imitates the kinematics of a natural knee.

According to a preferred embodiment the posterior recoil decreases between about 20 to 30° and 80 to 90°, an angle from which it becomes zero.

According to a preferred embodiment of the invention, the curve delimiting the transverse cross-section has at least two convexly shaped, in particular circular, segments meeting at a so-called summit point, the point formed by the perpendicular projection from the summit to a straight segment connecting the two convex segments being closer to the posterior end of the straight segment than to the anterior end, in particular by a ratio of ⅓ to ⅙.

According to a preferred embodiment of the invention the convex segments of the curve delimiting the transverse cross-section of the femoral stud correspond substantially to segments of the curve defined by the transverse cross-section in the antero-posterior or sagittal plane of the outer surface of the condyles, to within a homothetic relationship.

According to one development of the invention the tibial insert of the prosthesis having a tibial stud which projects, in particular in the perpendicular direction, from the base of the insert, and having a face intended to be turned towards the posterior side, is characterised in that in transverse cross-section in the sagittal plane, the posterior face defines a curve, a so-called contact curve, which is in the from of a concave curve with its concavity turned towards the posterior side, one point on the curve, in particular the summit of the curve, i.e. the point furthest from the base, being more posterior than other points on the curve, in particular most of the other points on the curve.

In this prosthesis the femoral stud rolls or slides on the posterior face of the tibial stud as the knee flexes. In current prostheses the point of contact between the femoral stud and the tibial stud tends to rise (i.e. to move away from the base of the insert) as the knee flexes. By thus providing a more posterior summit for the contact curve, possible unhooking at large flexion angles is counteracted and a safer prosthesis is thus obtained.

According to a preferred embodiment of the invention the contact curve has a straight segment, in particular in its intermediate part between the base and the summit.

According to one development of the invention the curve is at least partially of such a shape that from a given point to the summit a point on the curve is more posterior the closer it is to the summit.

The point of contact between the femoral stud and the tibial stud will tend to descend as flexion progresses, thus reducing the risk of shifting.

A preferred embodiment of the invention will now be described given solely by way of example and with reference to the drawings in which:

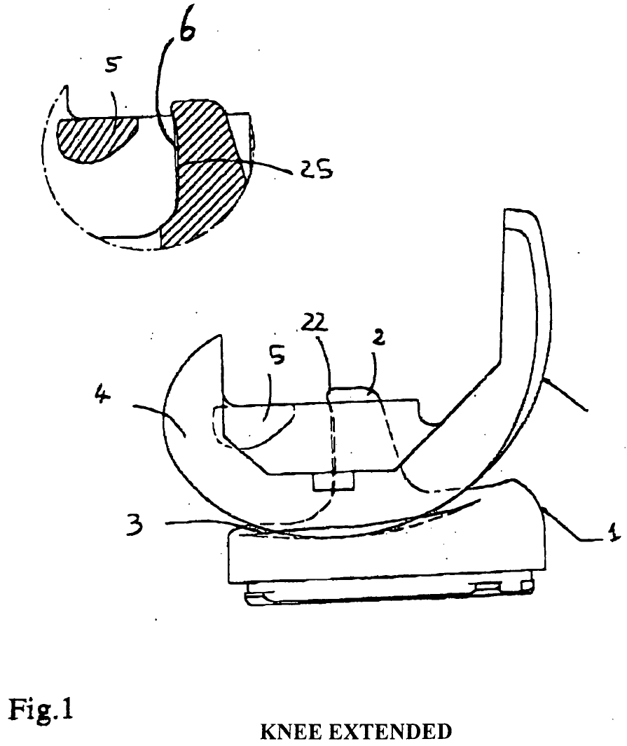

FIG. 1 illustrates the femoral part of a postero-stabilised prosthesis on a tibial insert intended to be placed on a tibial plate in the so-called antero-posterior plane when the knee is in the extended position (flexion at 0°);

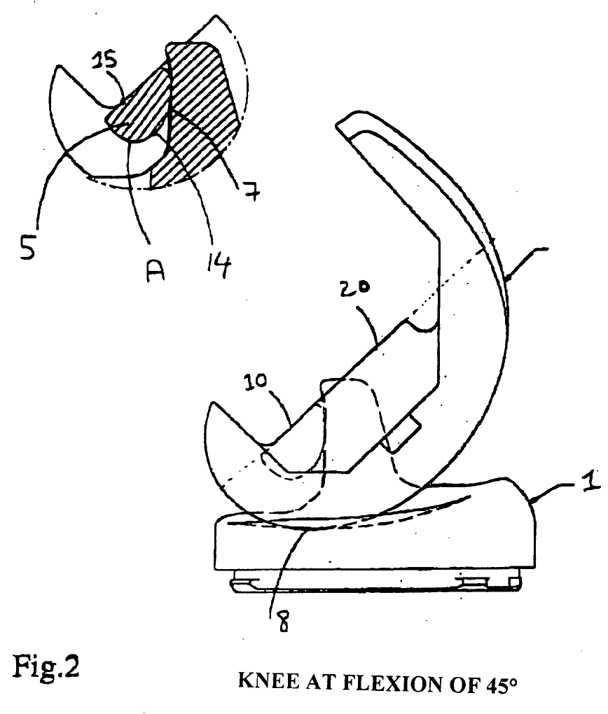

FIG. 2 illustrates the femoral part of FIG. 1 for a flexion of 45°;

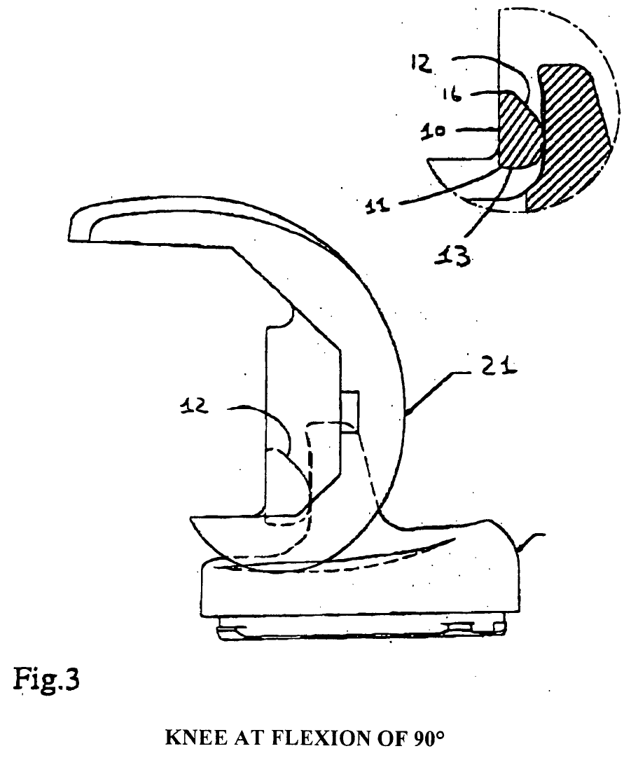

FIG. 3 illustrates the femoral part of FIG. 1 for a flexion of 90°;

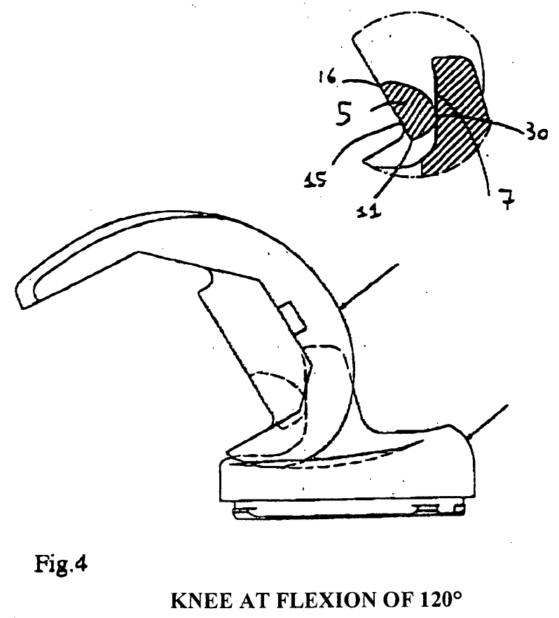

FIG. 4 illustrates the femoral part of FIG. 1 for a flexion of 120°.

FIG. 1 shows, in the antero-posterior plane, i.e. in the plane defined by the longitudinal axes of the femur and of the tibia during flexion, a tibial insert 1 made from polyethylene, having a tibial stud 2 projecting in the vertical direction from a base of the insert, the base having two upper contact surfaces 3 with which the outer surfaces of the condyles 4 are in contact.

Between the two right and left condyles 4 (only one is shown in the figures which are cross-sectional views) an inter-condyle space is formed through which the tibial stud 2 passes. A femoral stud 5 extends from one condyle to the other in the medio-lateral direction (perpendicular to the plane of the figures).

When the knee is in the extended position, the tibial stud and the femoral stud are spaced apart from each other. From a flexion of 30° the femoral stud comes into contact with a posterior face 6 of the tibial stud. Each condyle is in contact with the insert at a contact zone, having a central point 8. In some cases, depending on the relative geometries of the contacting surfaces, this zone can simply be a point which will then also be the central point.

According to the invention the shape of the transverse cross-section of the femoral stud is selected by taking account of the shape of the condyles, the contact surfaces of the insert and of the posterior face of the tibial stud so that the point 8 is fixed for any flexion angle between 80 to 90° and 130°.

Starting from a given shape for the concave upper surfaces of the insert, from a given shape for the outer surfaces of the condyles and from the shape of the posterior face of the tibial stud, it is possible to determine, point by point, the shape of the curve of the transverse cross-section of the femoral stud.

Thus taking the geometries of the different elements of these figures, the curve A has been obtained.

In the case where the condyles are formed by two circular surfaces with radii R1 and R2 (see figures) good results are obtained by providing a curve A having two circular curves of radii R1/H and R2/H, where H is a coefficient of homothecy, which is dependent upon the overall size of the prosthesis.

It is thus possible to make provision for the transverse cross-section of the femoral stud to be formed by a straight segment 10 having a posterior end 11 and an anterior end 16, from which ends two segments of curves 12 and 13, posterior and anterior, extend. The two segments of curves are convex, for example circular. They meet at a summit 14. The perpendicular projection 15 from the summit 14 to the straight section 10 is located closer to the posterior end 11 than to the anterior end 16. In particular the ratio of the distance from 15 to 11 with respect to the distance from 15 to 16 is between ⅓ and ⅙.

In particular, as shown by the figures, the shape of the closed curve 10-12-13 corresponds, to within a homothetic relationship, to the shape of the outer surface 21 of the condyles below the horizontal when the knee is in the extended position supplemented by a horizontal segment 20 (partially shown in dashed lines in the figure).

At its summit the tibial stud has a sort of tip 22 projecting in the direction of the femoral stud. The posterior surface of the femoral stud is dish-shaped, the bottom 25 of the dish being substantially flat and extending over substantially the whole vertical extent of the stud.

As also shown by the figures, the shape of the posterior surface of the tibial stud is selected in such a way that the point of contact 30 between the femoral stud and the tibial stud descends as the angle of flexion increases. The risk of shifting of the prosthesis is thus further reduced.

1. Full knee prosthesis, a so-called postero-stabilised prosthesis, formed by a femoral part comprising two condyles between which extends a femoral stud of a cylindrical shape in order to define an opening, by a tibial part having a tibial plate, and by a tibial insert disposed between the femoral part and the tibial plate, a tibial stud projecting from a piece of the tibial insert, passing into the opening and coming into contact with the femoral stud from a given angle, in particular between about 20 and 30°, the tibial insert having concave upper surfaces in contact with the convex outer surfaces of the condyles, the contact defining a contact zone having a central point, is characterised in that the curve delimiting the transverse cross-section of the femoral stud is defined so that the central point of the contact zone undergoes a translation or recoil in the posterior direction for any angle of flexion between the said given angle up to 80 to 90°, and no longer undergoes any posterior recoil for any angle between about 80 to 90° and a maximum flexion of 120-130°.

2. Full knee prosthesis as claimed in claim 1, characterised in that the posterior recoil decreases between about 20 to 30° and 80 to 90°, an angle from which it becomes zero.

3. Full knee prosthesis as claimed in claim 1, characterised in that the curve delimiting the transverse cross-section has two segments (12, 13) which are convex, in particular arcs of a circle, meeting at a so-called summit point (14), the point formed by the perpendicular projection from the summit to a straight segment connecting the two convex segments being closer to the posterior end of the straight segment.

4. Full knee prosthesis as claimed in claim 3, characterised in that the convex segments (12, 13) of the curve delimiting the transverse cross-section of the femoral stud (5) correspond substantially to segments of the curve defined by the transverse cross-section in the antero-posterior plane of the outer surface (21) of the condyles.

5. Prosthesis as claimed in claim 1, the tibial insert (1) of the prosthesis having a tibial stud (2) projecting, in particular in the perpendicular direction, from the base of the insert, and having a face (6) intended to be turned towards the posterior side, characterised in that in transverse cross-section in the sagittal plane, the posterior face (6) defines a curve, a so-called contact curve, which is in the form of a concave curve with its concavity turned towards the posterior side, the summit of the curve, i.e. the point furthest from the base, being more posterior than other points on the curve, in particular most of the other points on the curve.

6. Prosthesis as claimed in claim 5, characterised in that the contact curve has a straight segment, in particular in its intermediate part between the base and the summit.

7. Prosthesis as claimed in claim 6, characterised in that the curve is at least partially of such a shape that from a given point up to the summit a point on the curve is more posterior the closer it is the summit.

8. Femoral part of a knee prosthesis having two condyles between which an inter-condyle space is defined, delimited on the posterior side by a femoral stud (5) extending in the medio-lateral direction, characterised in that the curve delimiting the transverse cross-section has two segments (12, 13) which are convex, in particular arcs of a circle, meeting at a so-called summit point (14), the point formed by the perpendicular projection from the summit to a straight segment connecting the two segments being closer to the posterior end of the straight segment than to the anterior end, in particular by a ratio of ⅓ to ⅙.

9. Femoral part as claimed in claim 8, characterised in that the segments (12, 13) which are convex, in particular arcs of a circle, of the curve delimiting the transverse cross-section of the femoral stud (5) correspond substantially to segments of the curve defined by the transverse cross-section in the antero-posterior of sagittal plane of the outer surface (21) of the condyles (4), to within a homothetic relationship.
